(19) United States
(12) Patent Application Publication
(10) Pub. No.: US 2023/0270644 A1
(43) Pub. Date: Aug. 31, 2023

(54) COMPOSITION FOR CLEANSING AND/OR REMOVING MAKEUPS FROM KERATIN MATERIALS

(71) Applicant: L'OREAL, Paris (FR)

(72) Inventors: Yong WANG, Shanghai (CN); Chunyue LIU, Shanghai (CN)

(73) Assignee: L'OREAL, Paris (FR)

(21) Appl. No.: 18/006,811

(22) PCT Filed: Sep. 23, 2020
(86) PCT No.: PCT/CN2020/116942

§ 371 Date: Jan. 25, 2023

(51) Int. Cl. A61K 8/44; A61K 8/34; A61K 8/365; A61K 8/46; A61Q 1/14

(52) U.S. Cl. CPC A61K 8/44; A61K 8/345; A61K 8/365; A61K 8/466; A61Q 1/14

(57) ABSTRACT

Disclosed is a composition for cleansing and/or removing makeups from keratin materials, comprising in an aqueous phase: a) at least one amino acid surfactant; b) at least one sulfosuccinate surfactant; and c) at least one divalent and/or trivalent metal salt. Also disclosed is a process for cleansing and/or removing makeups from keratin materials, in particular the skin, comprising the application to the keratin materials, in particular the skin, of the composition according to the present invention, and rinsing off said composition after an optional period of time.

### TECHNICAL FIELD

The present invention relates to a cosmetic composition, in particular, a composition for cleansing and/or removing makeups from keratin materials. The present invention also relates to a process for cleansing and/or removing makeups from keratin materials using said composition.

### BACKGROUD ART

Cleansing the skin or removing makeups from the skin is very important for caring for the skin. It must be as efficient as possible because greasy residues, such as excess sebum, the remnants of cosmetic products used daily and make-up products, in particular waterproof products, accumulate in the skin folds, and can block the pores of the skin and result in the appearance of spots.

Several types of skin cleansing or makeup removing products, for example, rinsable anhydrous oils and gels, foaming creams and lotions, are known.

Rinsable anhydrous oils and gels have a cleansing or makeup removing action by virtue of oils present in these formulations. These oils make it possible to dissolve fatty residues and to disperse make-up pigments. These products are effective and well tolerated.

However, they exhibit the disadvantages of being heavy, of not foaming and of not conferring a good skin finish upon application, which is disadvantageous from a cosmetic viewpoint.

On the other hand, foaming creams, lotions and gels have a cleansing or makeup removing action by virtue of the surfactants, which suspend the fatty residues on the face. They are effective and pleasant to use because they foam and they are easy to remove.

In addition, for some lotions, there is dripping issue due to the low viscosity.

Amino acid surfactants are commonly used in skin cleansing or makeup removing products due to their mildness. However, the rinsibility is not satisfying.

Furthermore, for some commercial cleanser, the rinse-off speed is not quick, which brings unpleasant usage.

Therefore, there is a need to formulate compositions for cleansing or removing makeups from keratin materials, which are mild and have improved foaming ability, can provide a quick rinse-off speed and a good skin finish, and do not have dripping issue.

### SUMMARY OF THE INVENTION

The inventors have found that such a need can be achieved by the present invention.

Thus, according to an aspect, the present invention relates to a composition for cleansing and/or removing makeups from keratin materials, comprising in an aqueous phase:
a) at least one amino acid surfactant;
b) at least one sulfosuccinate surfactant; and
c) at least one divalent and/or trivalent metal salt.

The composition according to the present invention presents a paste, cream or texturized lotion aspect, and there is no dripping issue.

The composition according to the present invention also provides a quick rinse-off speed and a good foaming volume as well as no-irritation feeling during application.

The composition according to the present invention further provides a good skin finish (i.e. a skin feeling not tight and not dry) after application.

It may be used as a daily facial cleanser and/or makeup remover. The composition of the present invention is a rinse-off product. Thus, such a composition can be applied on the skin (i.e. face and/or body), and then rinsed with flush water.

According to another aspect, the present invention provides a process for cleansing and/or removing makeups from keratin materials, in particular the skin, comprising the application to the keratin materials, in particular the skin, of the composition according to the present invention, and rinsing off said composition after an optional period of time.

Other subjects and characteristics, aspects and advantages of the invention will emerge even more clearly on reading the description and the examples that follow.

### BRIEF DESCRIPTION OF THE DRAWINGS

Implementations of the present invention will now be described, by way of example only, with reference to the attached figures, wherein:

FIG. 1 provides an illustration of the test for determining the dripping speed.

### DETAILED DESCRIPTION OF THE INVENTION

In that which follows and unless otherwise indicated, the limits of a range of values are included within this range, in particular in the expressions “between . . . and . . . ” and “ranging from . . . to . . . ”.

For the purposes of the present invention, the term “keratin material” is intended to cover human skin, mucous membranes such as the lips. Facial skin is most particularly considered according to the present invention.

Moreover, the expression “at least one” used in the present description is equivalent to the expression “one or more”.

Throughout the instant application, the term “comprising” is to be interpreted as encompassing all specifically mentioned features as well optional, additional, unspecified ones.

As used herein, the use of the term “comprising” also discloses the embodiment wherein no features other than the specifically mentioned features are present (i.e. “consisting of”).

According to an aspect, the present invention relates to a composition for cleansing and/or removing makeups from keratin materials, comprising, in an aqueous phase:
a) at least one amino acid surfactant;
b) at least one sulfosuccinate surfactant; and
c) at least one divalent and/or trivalent metal salt.

Amino Acid Surfactants

The composition according to the present invention comprises at least one amino acid surfactant.

In one embodiment, said amino acid surfactant is derived from a carboxylate salt of amino acid wherein the amino group situated on the α-carbon or β-carbon of an amino acid salt is acylated with a C₈-C₂₂ fatty acid derivative.

The carboxylate salts of these amino acids can be formed by conventional means such as by neutralization of the respective amino acid with a base. The amino group situated on the α-carbon or β-carbon of the neutralized amino acid is acylated with a fatty acid halide (acyl halide) in the presence of a base via the well-known Schotten-Baumann reaction giving the amide, thus forming the desired surfactant reaction product, i.e. the amino acid surfactant. Suitable acyl halides for acylation of the amino acid carboxylate salt include acyl chlorides, bromides, fluorides, and iodides. The acyl halides can be prepared by reacting a saturated or unsaturated, linear or branched C₈-C₂₂ fatty acid with a thionyl halide (bromide, chloride, fluoride, and iodide). Representative acyl halides include but are not limited to the acyl chlorides selected from decanoyl chloride, dodecanoyl chloride (lauroyl chloride), cocoyl chloride (coconut oil derived fatty acid chlorides) tetradecanoyl chloride (myristoyl chloride), hexadecanoyl chloride (palmitoyl chloride), octadecanoyl chloride (stearoyl chloride), 9-octadecenoyl chloride (oleoyl chloride), eicosanoyl chloride (arachidoyl chloride), docosanoyl chloride (behenoyl chloride), and any mixture thereof. Other acyl halides include the bromides, fluorides and iodides of the foregoing fatty acids. A method for preparing acyl halides as well as an alternative method for acylating amino acids is set forth in US Patent Application Publication No. 2008/0200704, published on Aug. 21, 2008, which application is incorporated herein by reference.

Preferably, said amino acid surfactant is represented by the formula (I):

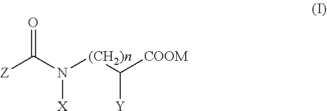

wherein:
Z represents a saturated or unsaturated, linear or branched hydrocarbon group having 8 to 22 carbon atoms,
X is hydrogen or methyl group,
n is 0 or 1,
Y is selected from hydrogen, —CH₃, —CH(CH₃)₂, —CH₂CH(CH₃)₂, —CH(CH₃)CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)O⁻M⁺, —(CH₂)₂C(O)OH, —(CH₂)₂C(O)O⁻M⁺, and
M is a salt-forming cation wherein COO is the counter-anion, such as for example sodium, potassium, ammonium, or triethanolamine.

In one embodiment, in formula (I):
Z represents a saturated or unsaturated, linear or branched C₈-C₂₂ alkyl group,
X is a hydrogen or methyl group,
n is 0,
Y is selected from hydrogen, —(CH₂)₂C(O)OH, —(CH₂)₂C(O)O⁻M⁺, and
M is a salt-forming cation wherein COO is the counter-anion, such as sodium, potassium, ammonium, or triethanolamine.

According to a preferred embodiment of the invention, in formula (I):
Z represents a saturated or unsaturated, linear or branched C₈-C₂₂ alkyl group,
X is a hydrogen or methyl group,
n is 0,
Y is selected from hydrogen, —(CH₂)₂C(O)OH, —(CH₂)₂C(O)O⁻M⁺, and
M is a salt-forming cation wherein COO is the counter-anion, such as for example sodium, potassium, ammonium, or triethanolamine.

Examples of the amino acid surfactants are salt of alanine, arginine, aspartic acid, glutamic acid, glycine, isoleucine, leucine, lysine, phenylalanine, serine, tyrosine, valine, sarcosine, and any mixture thereof. More specifically, mentions can be made of the amino acid surfactants such as dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, cocoyl methyl β-alaninate, lauroyl β-alaninate, lauroyl methyl β-alaninate, myristoyl β-alaninate, potassium lauroyl methyl β-alaninate, sodium cocoyl alaninate, sodium cocoyl methyl β-alaninate and sodium myristoyl methyl β-alaninate palmitoyl glycinate, sodium lauroyl glycinate, sodium cocoyl glycinate, sodium myristoyl glycinate, potassium lauroyl glycinate, potassium cocoyl glycinate, potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate ammonium lauroyl sarcosinate, sodium lauroyl aspartate, sodium myristoyl aspartate, sodium cocoyl aspartate, sodium caproyl aspartate, disodium lauroyl aspartate, disodium myristoyl aspartate, disodium cocoyl aspartate, disodium caproyl aspartate, potassium lauroyl aspartate, potassium myristoyl aspartate, potassium cocoyl aspartate, potassium caproyl aspartate, dipotassium lauroyl aspartate, dipotassium myristoyl aspartate, dipotassium cocoyl aspartate, dipotassium caproyl aspartate, and mixtures thereof.

References can be made to the commercially available amino acid surfactant of, for example, acylsarcosinates, for instance the sodium lauroyl sarcosinate sold under the name Sarkosyl NL 97® by the company Ciba or sold under the name Oramix L 30® by the company SEPPIC, the sodium myristoyl sarcosinate sold under the name Nikkol Sarcosinate MN® by the company Nikkol or the sodium palmitoyl sarcosinate sold under the name Nikkol Sarcosinate PN® by the company Nikkol; alaninates, for instance the sodium N-lauroyl-N-methylamidopropionate sold under the name Sodium Nikkol Alaninate LN 30® by the company Nikkol or sold under the name Alanone ALE® by the company Kawaken, and the N-lauroyl-N-methylalanine triethanolamine sold under the name Alanone Alta® by the company Kawaken; N- acylglutamates, for instance the triethanolamine monococoylglutamate sold under the name Acylglutamate CT-12® by the company Ajinomoto and the triethanolamine lauroylglutamate sold under the name Acylglutamate LT-12® by the company Ajinomoto; glycinates, for instance sodium N-cocoylglycinate sold under the name Amilite GCS-12® by the company Ajinomoto; aspartates, for instance the mixture of triethanolamine N-lauroyl aspartate and of triethanolamine N-myristoylaspartate, sold under the name Asparack® by the company Mitsubishi; citrates, and any mixture thereof.

According to the present invention, the preferred amino acid surfactant is selected from sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, or a mixture thereof.

Mentions of the preferred amino acid surfactant in the composition which is commercially available can be made to sodium lauroyl sarcosinate (ORAMIX L 30 sold by Seppic), sodium cocoyl glycinate (and) water (AMILITE® GCS-12K sold by Ajinomoto), sodium cocoyl glutamate (and) disodium cocoyl glutamate (AMISOFT® CS-22 sold by Ajinomoto), and sodium lauroyl glutamate (AMISOFT° LS-11 sold by Ajinomoto).

According to a preferred embodiment, the amino acid surfactant is selected from sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, and a mixture thereof.

Due to the use of amino acid surfactant, the composition according to the present invention brings no irritation to keratin materials.

Advantageously, the amino acid surfactant is present in the composition according to the present invention in an amount ranging from 20 wt. % to 50 wt. %, preferably from 30 wt. % to 45 wt. %, relative to the total weight of the composition.

Sulfosuccinate Surfactants

The composition according to the present invention comprises at least one sulfosuccinate surfactant.

The sulfosuccinate surfactant can be oxyalkylenated and then preferably comprise from 1 to 50 ethylene oxide units, better from 1 to 10 ethylene oxide units.

The sulfosuccinate surfactants that are suitable for the invention can be selected from C₈-C₃₀ alkyl sulfosuccinates, C₈-C₃₀ alkylamide sulfosuccinates, optionally oxyalkylenated, and preferably, they are optionally oxyalkylenated C₈-C₃₀ alkyl sulfosuccinates, i.e. C₈-C₃₀ alkyl sulfosuccinates or C₈-C₃₀ alkylether sulfosuccinates.

The alkyl radical of the sulfosuccinate surfactant may comprise from 8 to 30 carbon atoms, preferably from 10 to 18 carbon atoms, and more preferentially from 10 to 16, better from 10 to 14.

As non-oxyalkylenated alkylsulfosuccinates, mention can be made of, sodium diethylhexyl sulfosuccinate such as the one marketed under the trade name TENSOMILD H026 by KLK OLEO, laurylic alcohol sulfosuccinates (C12/C14 70/30) (DISODIUM LAURYL SULFOSUCCINATE) such as the one marketed under the trade name Rewopol® SB F 12 P by Evonik Goldschmidt, KOHACOOL L-40 by TOHO CHEMICAL, MACKANATE LO-FF by RHODIA.

As oxyalkylenated sulfosuccinates, mention can be made of oxyethylenated laurylic alcohol sulfosuccinates (C12/C14 70/30) (DISODIUM LAURETH SULFOSUCCINATE) such as those that are marketed under the names SETACIN 103 SPECIAL NP® by Zschimmer&Schwarz, REWOPOL SB FA 30 U by Evonik Goldschmidt, GOODWAY MES by SHANGHAI GOODWAY CHEMICAL, REWOPOL SB FA 30 PH by Evonik Goldschmidt, ALKONIX SS K by ULTRA-OXITENO, DISODIUM LAURETH SULFOSUCCINATE by GUANGZHOU FLOWER′S SONG FINE CHEMICAL, KOHACOOL L-300 by TOHO CHEMICAL, EMPICOL SDD OF by HUNTSMAN, disodium salt of an C12-C14 alcohol hemi-sulfosuccinate, marketed under the name SETACIN F SPECIAL PASTE® by Zschimmer&Schwarz, oxyethylene disodium oleamidosulfosuccinate (2 OE) marketed under the name STANDAPOL SH 135® by Cognis, lauric amide oxyethylene mono-sulfosuccinate (5 OE) marketed under the name LEBON A-5000® by Sanyo, disodium salt of lauryl citrate oxyethylene mono- sulfosuccinate (10 OE) marketed under the name REWOPOL SB CS 50® by Witco, ricinoleic mono-ethanolamide mono-sulfosuccinate marketed under the name REWODERM S 1333® by Witco. Polydimethylsiloxane sulfosuccinates such as disodium dimethicone sulfosuccinate PEG-12 marketed under the name MACKANATE-DC30® by the Mac Intyre Company can also be used.

According to a particular embodiment, the sulfosuccinate surfactant satisfies the following formula:

CH₃—(CH₂)n-(O—CH₂—CH₂)m-O—CO—CH(SO₃M)—CH₂—CO—OM′ (II)
wherein:
n designates an integer ranging from 7 to 29, preferably from 9 to 17, and more preferably from 9 to 15, most preferably from 9 to 13;
m designates an integer varying from 0 to 50,
M and M′ designate, independently of each other, a hydrogen atom, an alkali metal or alkali earth metal, an ammonium group or a cation derived from an amine.

When the sulfosuccinate surfactants are not oxyalkylenated, m is equal to 0.

When the sulfosuccinate surfactants are oxyalkylenated, m designates an integer ranging from 1 to 50, preferably from 1 to 10, and even more preferably from 1 to 8, most preferably from 1 to 6.

When the sulfosuccinates are in the form of salt, they can be selected from alkali metal salts such as sodium or potassium salt and preferably sodium, ammonium salts, amine salts and in particular aminoalcohols, alkaline earth metal salts such as magnesium salt. In this case, one or the other of the sulfonate functions, or both, can be found in the form of salt.

According to a particular embodiment of the invention, the sulfosuccinate is in the form of salt. Preferably, they are selected from alkali metal salts, and more particularly sodium salt. Preferably, they are di-salts of sodium.

According to a particular embodiment, the sulfosuccinate surfactant used in the composition of the present invention is an oxyalkylenated or non-oxyalkylenated alkylsulfosuccinate in which the alkyl radical comprises from 8 to 30 carbon atoms, preferably from 10 to 18, and more preferably from 10 to 16, preferably from 10 to 14, more preferably if it is a lauryl, diethylhexyl, and/or laurethsulfosuccinate, even more preferably a lauryl, diethylhexyl, and/or laurethsulfosuccinate alkali metal salt, and even more preferably disodium lauryl sulfosuccinate, sodium diethylhexyl sulfosuccinate, and/or disodium laureth sulfosuccinate.

Thus, according to a preferred embodiment, the sulfosuccinate surfactant is selected from disodium lauryl sulfosuccinate, sodium diethylhexyl sulfosuccinate, disodium laureth sulfosuccinate, a mixture thereof.

Preferably, the sulfosuccinate surfactant is present in the composition according to the present invention in an amount ranging from 0.5 wt. % to 20 wt. %, preferably from 1 wt. % to 15 wt. %, and more preferably from 3 wt. % to 6 wt. %, relative to the total weight of the composition.

Metal Salts

The composition according to the present invention comprises at least one divalent and/or trivalent metal salt.

The divalent and/or trivalent metal salt comprises a metal ion Mⁿ⁺. Preferably, Mⁿ⁺ is selected from Mg²⁺, Ca²⁺, Zn²⁺, Fe²⁺, Mn²⁺, Cu²⁺, Al³⁺ and Fe³⁺.

The metal salt is selected from metal salts of an organic acid or an inorganic acid.

As example of organic acid, mention can be made of ascorbic acid, formic acid, acetic acid, glycolic acid, gluconic acid, lactic acid, mandelic acid, oxalic acid, maleic acid, malonic acid, glyoxylic acid, succinic acid, adipic acid, fumaric acid, sebacic acid, citric acid, tartaric acid, malic acid, tricarboxylic acid, glutaric acid, glucaric acid, pyrrolidone carboxylic acid, phenol sulfonic acid, salicylic acid, etc.

As example of inorganic acid, mention can be made of sulfuric acid, carbonic acid, silicic acid, hydrochloric acid, nitric acid, phosphoric acid, etc.

Preferably, metal salts of an inorganic acid are selected from metal chlorides, sulfates, nitrates, carbonates and hydrogen carbonates, phosphates, silicates, and mixtures thereof, wherein the metal is selected from Mg, Ca, Zn, Mn, Cu, Al and Fe.

More preferably, metal salts of an inorganic acid are selected from calcium chloride, calcium sulfate, calcium nitrate, calcium carbonate and hydrogen carbonate, calcium phosphate, zinc chloride, zinc sulfate, zinc nitrate, zinc carbonate and hydrogen carbonate, zinc phosphate, magnesium chloride, magnesium sulfate, magnesium nitrate, magnesium carbonate and hydrogen carbonate, magnesium phosphate, magnesium aluminum silicate, aluminum calcium sodium silicate, and mixtures thereof.

Preferably, metal salts of an organic acid are selected from metal ascorbates, formates, acetates, glycolates, gluconates, lactates, mandelates, oxalates, maleates, malonates, glyoxylates, succinates, adipates, fumarates, sebacates, citrates, tartarates, malates, tricarboxylates, glutarates, glucarates, pyrrolidone carboxylates, phenolsulfonate, salicylates, and mixtures thereof, wherein the metal is selected from Mg, Ca, Zn, Mn, Cu, Al and Fe.

More preferably, metal salts of an organic acid are selected from magnesium gluconate, magnesium PCA (Magnesium Ascorbyl Phosphate), magnesium acetate, calcium PCA, zinc lactate, zinc gluconate, zinc phenolsulfonate, zinc salicylate, zinc pyrrolidone carboxylic acid (Zinc PCA), zinc citrate, zinc ascorbate, copper PCA, copper gluconate, and mixtures thereof.

According to a preferred embodiment, the metal salt is selected from magnesium gluconate, magnesium PCA, magnesium sulfate, magnesium acetate, magnesium carbonate, magnesium aluminum silicate, calcium carbonate, calcium PCA, aluminum calcium sodium silicate, calcium chloride, calcium carbonate, zinc PCA, zinc gluconate, copper PCA, copper gluconate, and a mixture thereof.

Advantageously, the metal salt is present in the composition according to the present invention in an amount ranging from 0.1 wt. % to 10 wt. %, preferably from 0.25 wt. % to 8 wt. %, more preferably from 0.5 wt. % to 5 wt. %, relative to the total weight of the composition.

Aqueous Phase

The composition of the present invention comprises at least one aqueous phase.

According to a preferred embodiment, the composition of the present invention is an aqueous paste.

The aqueous phase of the composition according to the present invention comprises water and optionally one or more water-miscible or at least partially water-miscible compounds, for instance C₂-C₈ polyols or monoalcohols, such as ethanol and isopropanol.

The term “polyol” should be understood as meaning any organic molecule comprising at least two free hydroxyl groups. Examples of polyols that may be mentioned include glycols, for instance butylene glycol, propylene glycol, dipropylene glycol, isoprene glycol, hexylene glycol, caprylyl glycol, glycerol (i.e. glycerin) and polyethylene glycols.

According to a preferred embodiment, the composition according to the present application further comprises at least one C₂-C₈ polyol selected from butylene glycol, propylene glycol, dipropylene glcol, isoprene glycol, hexylene glycol, caprylyl glycol, glycerin and polyethylene glycols.

With the presence of C₂-C₈ polyol, it is easy to form a crystalized system.

Preferably, the C₂-C₈ polyol is present in an amount ranging from 30 wt. % to 40 wt. %, relative to the total weight of the composition.

The aqueous phase may represent from 40 wt. % to 70 wt. %, preferably from 45 wt. % to 65 wt. %, relative to the total weight of the composition.

Additional Ingredients

The composition according to the present invention may comprises one or more additional ingredients, selected from those conventionally used in skincare cleasners.

The composition in accordance with the present invention may comprise any of the following additives: pH adjusting agents (e.g. citric acid); additional surfactants (e.g. amphoteric surfactant); biological extracts (e.g. RIBES NIGRUM (BLACK CURRANT) BUD EXTRACT); antibacterial agents, fragrances (e.g. perfumes, essential oils); thickeners (e.g. glycol distearate); and cationic preservatives (e.g. myrtrimonium bromide.

A person skilled in the art can adjust the type and amount of additional ingredients present in the compositions according to the present invention by means of routine operations, so that the desired properties of these compositions are not adversely affected by the additional ingredients.

According to a preferred embodiment, the present invention relates to a composition for cleansing and/or removing makeups from keratin materials, comprising in an aqueous phase, relative to the total weight of the composition:
a) from 30 wt. % to 45 wt. % of at least one amino acid surfactant selected from sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, and a mixture thereof;
b) from 3 wt. % to 6 wt. % at least one sulfosuccinate surfactant selected from disodium lauryl sulfosuccinate, sodium diethylhexyl sulfosuccinate, disodium laureth sulfosuccinate, and a mixture thereof;
c) from 0.5 wt. % to 5 wt. % at least one divalent and/or trivalent metal salt selected from magnesium gluconate, magnesium PCA, magnesium sulfate, magnesium acetate, magnesium carbonate, magnesium aluminum silicate, calcium carbonate, calcium PCA, aluminum calcium sodium silicate, calcium chloride, calcium carbonate, zinc PCA, zinc gluconate, copper PCA, copper gluconate, and a mixture thereof; and
d) from 30 wt. % to 40 wt. % of at least one C₂-C₈ polyol selected from butylene glycol, propylene glycol, dipropylene glycol, isoprene glycol, hexylene glycol, caprylyl glycol, glycerin and polyethylene glycols.

Process and Use

The composition according to the present invention can be used in a process for cleansing and/or removing makeups from keratin materials, such as the skin, in particular the face, by being applied to the keratin materials.

The composition according to the present invention is a crystalized system in the form of a paste, cream or textured lotion.

There is no dripping issue for the composition according to the present invention.

By “no dripping issue”, it means the dripping speed of the composition is greater than 300 s.

As used herein, the dripping speed refers to the time that 90.0 g of sample to be tested takes to flow through an orifice with a diameter of 10 mm at 25.0° C., which can measured as follows.

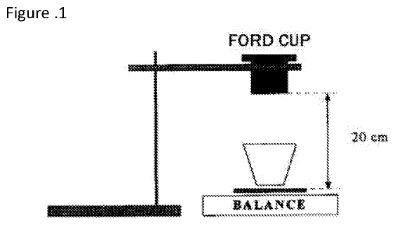

As shown in FIG. 1, a non-aerated sample of homogeneous appearance is introduced into a Ford cup (a stainless steel equipment comprising a body and an orifice with a diameter of 10 mm, the body and the orifice match perfectly). A thermostat is provided to maintain the sample and the Ford Cup at a temperature of 25.0° C. for a sufficient time. Then the Ford cup is placed on a horizontal surface, at a distance corresponding to a trickle height of 20 cm. A balance is placed in position to collect the sample and tare. The orifice is open and a stopwatch is started at the same time. The stopwatch is stop when the balance indicates 90.0±0.5 g. The test is considered valid only if 90 g sample flows in a continuous way.

The composition according to the present invention also provides a quick rinse-off speed and a good foaming volume as well as no-irritation feeling during application.

The composition according to the present invention further provides a good skin finish (i.e. a skin feeling not tight and not dry) after application.

The composition according to the invention may be applied by any means enabling a uniform distribution, in particular using a finger, or a cotton ball, and can be removed by rinsing with water.

Thus, according to another aspect, the present invention relates to a process for cleansing and/or removing makeups from keratin materials, in particular the skin, comprising the application to the keratin materials, in particular the skin, of the composition according to the present invention, and rinsing off said composition after an optional period of time.

The present invention is illustrated in greater detail by the examples described below, which are given as non-limiting illustrations.

The percentages are weight percentages by active ingredient.

In the examples that follow, the weight percentages are indicated relative to the total weight of the composition.

### EXAMPLES

### Comparative Example 1

The composition according to comparative formula 1 (comp. 1, a commercial cleanser) comprising the ingredients shown in Table 1 was prepared, with all amounts expressed by percentages by weight of active matter with regard to the total weight of each composition.

TABLE 1

| Ingredients | Content (wt. %) |
|---|---|
| SODIUM COCOYL GLYCINATE | 43.33 |
| GLYCERIN | 39.3 |
| WATER | QS100 |
| CITRIC ACID | 0.5 |
| STEARIC ACID | 1.1 |
| SODIUM CHLORIDE | 5 |

Preparation Process

The composition was prepared as follows.

Sodium cocoyl glycinate and glycerin were introduced into a main kettle and heated to 80° C. with stirring. Then water and citric acid were pre-mixed to obtain a mixture and the mixture was introduced into the main kettle with stirring. Next, stearic acid and sodium chloride were introduced in the main kettle one by one with stirring, to obtain the composition, which was then cooled down to room temperature.

### Invention Examples 1-4 and Comparative Examples 2-3

The compositions according to invention formulas (inv.) 1-4 and comparative formulas (comp.) 2-3 comprising the ingredients shown in Table 2 were prepared, with all amounts expressed by percentages by weight of active matter with regard to the total weight of each composition.

TABLE 2

| | Inv. 1 | Inv. 2 | Inv. 3 | Inv. 4 | comp. 2 | comp. 3 |
|---|---|---|---|---|---|---|
| Ingredients | Content(wt. %) | | | | | |
| SODIUM COCOYL GLYCINATE | 43.33 | 43.33 | 43.33 | 43.33 | 47.83 | 43.33 |
| GLYCERIN | 39.3 | 39.3 | 39.3 | 39.3 | 39.3 | 39.3 |
| WATER | QS100 | QS100 | QS100 | QS100 | QS100 | QS100 |
| CITRIC ACID | 0.25 | 0.25 | 0.25 | 0.25 | 0.25 | 0.25 |
| MAGNESIUM GLUCONATE | 0.5 | 1.5 | 2.5 | 5 | 2.5 | 0 |
| DISODIUM LAURYL | 4.5 | 4.5 | 4.5 | 4.5 | 0 | 4.5 |
| SULFOSUCCINATE | | | | | | |
| SODIUM CHLORIDE | 5 | 5 | 5 | 5 | 5 | 5 |

Preparation Process

Each composition was prepared as follows.

Sodium cocoyl glycinate and glycerin were introduced into a main kettle and heated to 80° C. with stirring. Water and citric acid were pre-mixed to obtain a mixture and the mixture was introduced into the main kettle with stirring. Magnesium gluconate, disodium lauryl sulfosuccinate and sodium chloride (if presents) were introduced in the main kettle one by one with stirring, to obtain the composition, which was then cooled down to room temperature.

### Comparative Example 4

The composition according to comparative formula 4 (comp. 4, a commercial cleanser) comprising the ingredients shown in Table 3 was prepared, with all amounts expressed by percentages by weight of active matter with regard to the total weight of each composition.

TABLE 3

| Ingredients | Content (wt. %) |
|---|---|
| SODIUM COCOYL GLYCINATE | 9 |
| GLYCERIN | 7 |
| LAURIC ACID | 1 |
| TRIETHANOLAMINE | 0.9 |
| COCO-BETAINE | 16 |
| PEG-120 METHYL GLUCOSE DIOLEATE | 2.5 |
| SODIUM CHLORIDE | 0.5 |
| DIPROPYLENE GLYCOL | 1 |
| WATER | QS100 |

Preparation Process

The composition was prepared as follows.

Sodium cocoyl glycinate and glycerin were introduced into a main kettle and heated to 80° C. with stirring. Lauric acid was introduced into the main kettle with stirring until it was totally dissolved or dispersed. Then triethanolamine was introduced into the main kettle slowly, followed by coco-betaine and PEG-120 methyl glucose dioleate. Sodium chloride, dipropylene glycol and water were introduced in the main kettle with stirring, to obtain the composition, which was then cooled down to room temperature.

### Comparative Example 5

The composition according to comparative formula 5 (comp. 5, a commercial cleanser) comprising the ingredients shown in Table 4 was prepared, with all amounts expressed by percentages by weight of active matter with regard to the total weight of each composition.

TABLE 4

| Ingredients | Content (wt. %) |
|---|---|
| WATER | QS100 |
| GLYCERIN | 21 |
| STEARIC ACID | 14 |
| PALMITIC ACID | 2 |
| MYRISTIC ACID | 12 |
| LAURIC ACID | 6 |
| GLYCERYL DISTEARATE (and) | 3 |
| GLYCERYL STEARATE | |
| KAOLIN | 3 |
| POTASSIUM HYDROXIDE | 13.8 |
| PEG-14M | 0.05 |

Preparation Process

The composition was prepared as follows.

Glycerin and PEG-14M were introduced into a main kettle and heated to 80° C. with stirring. Next, stearic acid, palmitic acid, myristic acid, lauric acid and glyceryl distearate (and) glyceryl stearate were introduced into the main kettle with stirring until they were totally dissolved or dispersed. Then water and potassium hydroxide were pre-mixed to obtain a mixture and the mixture was introduced into the main kettle slowly to neutralize the fatty acids. The mixture obtained was stirred at 80° C. over 30 minutes, then cooled down to 30° C. and kaolin was introduced into the mixture with stirring to obtain the composition.

### Invention Example 5

The composition according to invention formula (inv.) 5 comprising the ingredients shown in Table 5 was prepared, with all amounts expressed by percentages by weight of active matter with regard to the total weight of each composition.

TABLE 5

| Ingredients | Content (wt. %) |
|---|---|
| WATER | QS100 |
| GLYCERIN | 34.00 |
| SODIUM COCOYL GLYCINATE | 34.00 |
| MAGNESIUM GLUCONATE | 1 |
| DISODIUM LAURYL SULFOSUCCINATE | 4.5 |
| CITRIC ACID | 0.25 |
| COCO-BETAINE | 5 |
| SODIUM CHLORIDE | 5 |
| HYDROXYPROPYL STARCH PHOSPHATE | 1 |

Preparation Process

The composition was prepared as follows.

Glycerin and sodium cocoyl glycinate were introduced into a main kettle and heated to 80° C. with stirring. Next, magnesium gluconate and disodium lauryl sulfosuccinate were introduced into the main kettle one by one with stirring until they were totally dissolved or dispersed. Then water and citric acid were pre-mixed to obtain a mixture and the mixture was introduced into the main kettle. Next, coco-betaine, sodium chloride and hydroxypropyl starch phosphate were introduced into the main kettle one by one to obtain the composition, which was then cooled down to room temperature.

Evaluation of Compositions

The aspect of compositions of invention and comparative formulas were observed. The compositions were also evaluated in terms of eye Irritation, rinse off speed, skin finish, foaming property, and dripping speed, wherein the dripping speed was evaluated as mentioned previously.

Eye Irritation

Eye irritation was evaluated by a BCOP test according to OECD guideline 437 (OECD. 2009. OECD, Test No. 437: Bovine corneal opacity and permeability test method for identifying i) chemicals inducing serious eye damage and ii) chemicals not requiring classification for eye irritation or serious eye damage. Paris, France: OECD Publishing.)

Expression of Results:

To increase the classification of the test, the exposure time fixed at 30 minutes, the testing formulas are categorized into 4 classes, non-slightly irritant, slightly-moderately irritant, moderately irritant-irritant and irritant-severely irritant based on the following criterion.

| Score | Category |
|---|---|
| 0 < IVIS 30 min < 10 | No irritant to slightly irritant |
| 10 < IVIS 30 min < 25 | Moderately irritant to irritant |
| 25 < IVIS 30 min < 55 | Irritant to severely irritant |
| IVIS 30 min > 55 | Severely irritant |

Rinse Off Speed and Skin Finish

Preparation: taking 0.5 ml of the composition to be test with a syringe, and 1 ml of water with a pipette.

Procedures:
1) Rinse hands under tap water for 2 seconds twice, and dampen the face with wet hands each time;
2) Place 0.5 ml of the composition to be tested on to one palm, add 1 ml of water with the pipette;
3) Rub for 5 seconds between palms (4 circles/second), pause when necessary to gather foam in the palms to prevent from dripping;
4) Spread foam on both hands;
5) Apply the foam to the face (with palm and fingers), avoiding eye contour; and
6) Massage on face with the foam.

Rinse-off speed is used to define quickness of the strongest squeaky feeling of whole face occurs and was tested as follows:

Gesture: Speed of squeaky feel occurring is evaluated tactilely during 15 rinsing movements. A score within a range of 1-15 is given wherein 1-4 means a low rinse-off speed, 5-9 means a middle rinse-off speed and 10-15 means a high rinse-off speed.

Procedures:
a) Rinse hands under tap water for 2 seconds, move the wet hands (do not reserve water in palms) on the face from the chin to the forehead and back to the chin (2 sides of the face);
b) Repeat this rinsing procedure 15 times; and
c) Dry hands with a dry towel.

Skin finish was evaluated in terms of dry skin and tight skin.

Dry skin indicates a state of dehydration or relative deficiency of water.

Gesture: This attribute is evaluated visually by lifting cheeks by fingers, and observing the depth and the amount of dry lines on the face (above the cheeks under eyes). The higher score means the skin is not dry.

Tight skin indicates a tugging sensation.

Gesture: This attribute is evaluated by feeling absence or presence of tugging of skin when making facial expressions. A score within a range of 1-15 is given wherein 1-4 means a very dry and tight skin finish, 5-9 means a little dry and tight skin finish, 10-15 means a non-dry and non-tight skin finish.

Procedures:

Dry the face with a dry towel: gently place (without wiping or rubbing) the dry towel on the face to absorb the water, from the chin, cheeks to the forehead, once in each part, then wait for 2 minutes to evaluate dry skin and tight skin.

Foaming Property

Preparation: taking 0.5 ml of the composition to be tested with a syringe, 3×1 ml of water with pipettes.

Procedure:
1) Rinse hands under tap water for 2 seconds and place the 0.5 ml of product to palm with the syringe, add 1 ml of water with one pipette;
2) Make back and forth movements for 20 rounds (2 circles/second) and pause to gather foam in the palm to prevent foam from flowing if necessary;
3) Add 1 ml of water with one pipette again, and make back and forth movements for 20 rounds (2 circles/second), and pause to gather foam in the palm to prevent foam from flowing if necessary; and
4) Put together all the foam in one palm.

Foaming property is evaluated by the volume of foam obtained at the end of rubbing hands (40 rounds), regardless of size of bubbles. A score within a range of 0-15 is given wherein 0 means the volume of foam is zero, 1-4 means low foam volume, 5-9 means a middle foam volume and 10-15 means high foam volume.

The results of the evaluations are as follows:

| | | | Rinse-off | Foaming | Skin | Dripping |
|---|---|---|---|---|---|---|
| | | | speed | property | finish | speed |
| | Aspect | Eye irritation | (score) | (score) | (score) | (s) |
| Comp. 1 | White | No irritant to slightly | 10 | 8 | 10 | >600 |
| | Paste | irritant | | | | |
| Inv. 1 | White | No irritant to slightly | 12 | 14 | 12 | 325 |
| | Paste | irritant | | | | |
| Inv. 2 | White | No irritant to slightly | 13 | 15 | 13 | >600 |
| | Paste | irritant | | | | |
| Inv. 3 | White | No irritant to slightly | 14 | 12 | 11 | >600 |
| | Paste | irritant | | | | |
| Inv. 4 | White | No irritant to slightly | 14 | 10 | 10 | >600 |
| | Paste | irritant | | | | |
| Comp. 2 | White | No irritant to slightly | 13 | 8 | 11 | >600 |
| | Paste | irritant | | | | |
| Comp. 3 | White | No irritant to slightly | 9 | 11 | 11 | >600 |
| | Paste | irritant | | | | |
| Comp. 4 | Translucent | Moderately irritant to | 6 | 8 | 12 | 20 |
| | Gel | irritant | | | | |
| Comp. 5 | White | Moderately irritant to | 15 | 10 | 5 | >600 |
| | Paste | irritant | | | | |
| Inv. 5 | White | No irritant to slightly | 13 | 13 | 13 | >600 |
| | Paste | irritant | | | | |

From above results, it can be seen that compositions according to the present invention have no dripping issue, provide a quick rinse-off speed and a good foaming volume as well as no-irritation feeling during application, and provide a good skin finish after application.

A consumer test shows that compositions according to invention formulas 1-5 have good rinsibility.

1. A composition for cleansing and/or removing makeups from keratin materials, comprising in an aqueous phase:
a) at least one amino acid surfactant;
b) at least one sulfosuccinate surfactant; and
c) at least one divalent and/or trivalent metal salt.

2. The composition according to claim 1, wherein the amino acid surfactant is represented by the formula (I): 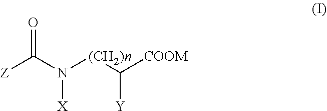
wherein:
Z represents a saturated or unsaturated, linear or branched hydrocarbon group having 8 to 22 carbon atoms,
X is hydrogen or methyl group,
n is 0 or 1,
Y is selected from hydrogen, —CH3, —CH(CH₃)₂, —CH₂CH(CH₃)₂, —CH(CH₃) CH₂CH₃, —CH₂CH₆H₅, —CH₂C₂H₄OH, —CH₂OH, —CH(OH)CH₃, —(CH₂)₄NH₂, —(CH₂)₃NHC (NH)NH₂, —CH₂C(O)O⁻M⁺, —(CH₂)₂C(O)OH, −(CH₂)₂C(O)O⁻M⁺, and
M is a salt-forming cation wherein COO is the counter-anion.

3. The composition according to claim 1, wherein the amino acid surfactant is selected from the group consisting of sodium lauroyl sarcosinate, sodium cocoyl glycinate, sodium cocoyl glutamate, sodium stearoyl glutamate, disodium cocoyl glutarn.ate, sodium lauroyl glutamate, potassium cocoyl glycinate, TEA-cocoyl glutamate, and a mixture thereof.

4. The composition according to claim 1, wherein the amino acid surfactant is present in the composition in an amount ranging from 20 wt. % to 50 wt. % relative to the total weight of the composition.

5. The composition according to claim 1, wherein the sulfosuccinate surfactant is selected from C₈-C₃₀alkyl sulfosuccinates, C₈-C₃₀alkylamide sulfosuccinates, optionally oxyalkylenated.

6. The composition according to claim 5, wherein the sulfosuccinate surfactant is selected from the group consisting of disodium lauryl sulfosuccinate, sodium diethylhexyl sulfosuccinate, disodium laureth sulfosuccinate, and a mixture thereof.

7. The composition according to claim 1, wherein the sulfosuccinate surfactant is present in the composition in an amount ranging from 0.5 wt. % to 20 wt. % relative to the total weight of the composition.

8. The composition according to claim 1, wherein the metal salt comprises a metal ion Mⁿ⁺ selected from the group consisting of Mg²⁺, Ca²⁺, Fe²⁺, Mn²⁺, Cu²⁺, Al³⁺ and Fe³⁺.

9. The composition according to claim 1, wherein the metal salt is selected from the group consisting of magnesium gluconate, magnesium ascorbyl phosphate (PCA), magnesium sulfate, magnesium acetate, magnesium carbonate, magnesium aluminum silicate, calcium carbonate, calcium PCA, aluminum calcium sodium silicate, calcium chloride, calcium carbonate, zinc PCA, zinc gluconate, copper PCA, copper gluconate, and a mixture thereof.

10. The composition according to claim 1, wherein the metal salt is present in the composition in an amount ranging from 0.1 wt. % to 10 wt. % relative to the total weight of the composition.

11. The composition according to claim 1, further comprising at least one C₂-C₈ polyol selected from the group consisting of hutylene glycol, propylene glycol, dipropylene glycol, isoprene glycol, hexylene glycol, caprylyl glycol, glycerin and polyethylene glycols.

12. The composition according to claim 11, wherein the C₂-C₈ polyol is present in an amount ranging from 30 wt. % to 40 wt. %, relative to the total weight of the composition.

13. The composition according to claim 1, wherein the aqueous phase represents from 40 wt. % to 70 wt. % relative to the total weight of the composition.

14. The composition according to claim 1, wherein the composition is a crystalized system in the form of a paste, cream or textured lotion.

15. A process for cleansing and/or removing makeups from keratin materials, comprising the application to the keratin materials, of the composition according to claim 1, and rinsing off said composition after an optional period of time.

16. The composition according to claim 1, wherein
Z represents a saturated or unsaturated, linear or branched C₈-C₂₂ alkyl group,
X is a hydrogen or methyl group,
n is 0,
Y is selected from hydrogen, —(CH₂)₂C(O)OH, —(CH₂)₂C(O)O⁻M⁺, and
M is sodium, potassium, ammonium, or triethanolamine.
